(19) United States
(12) Patent Application Publication
(10) Pub. No.: US 2023/0277352 A1
(43) Pub. Date: Sep. 7, 2023

(54) DYNAMIC FOOT ABDUCTION BAR

(71) Applicant: THE HOSPITAL FOR SICK CHILDREN, Toronto (CA)

(72) Inventors: Hannah Ruth Seatle, Oakville (CA); Maryse Lorraine Bouchard, Toronto (CA); Barbara Joyce Harvey, Toronto (CA)

(21) Appl. No.: 18/117,233

(22) Filed: Mar. 3, 2023

Related U.S. Application Data
(60) Provisional application No. 63/316,130, filed on Mar. 3, 2022.

(51) Int. Cl. A61F 5/01

(52) U.S. Cl. CPC A61F 5/0116

(57) ABSTRACT

A dynamic foot abduction bar for use with a pair of boots having a crossbar and a pair of generally L-shaped components. The generally L-shaped components are slidingly attached at either end of the crossbar, thereby allowing axial translation. The pair of boots are releasably attachable to the generally L-shaped components. The dynamic foot abduction bar may include a modified ball joint attached between the generally L-shaped component and the crossbar. The modified ball joint may be configured for upward coronal rotation, outward axial rotation and sagittal rotation.

### BACKGROUND

The present disclosure relates to orthotic devices and in particular a dynamic foot abduction bar for use in the treatment of clubfoot.

Clubfoot is one of the most common serious congenital deformities of the musculoskeletal system in newborns. The typical appearance of clubfoot is the foot pointing downwards and twisted inwards.

The standard of care for treatment of clubfoot is the Ponseti method. This consists of serial casting and a percutaneous Achilles tenotomy to correct the foot shape. The correction is maintained using a foot abduction brace in early childhood. Typical wear schedule for the foot abduction brace is 23 hours per day for three months, then at night for four years.

Compliance with corrective foot orthosis is important for a positive outcome. If the corrective foot orthosis is not worn, reoccurrence generally occurs. The main reasons for noncompliance with corrective foot orthosis is that they limit the mobility of the patient's legs, are uncomfortable, and if not fitted well, can form blisters/pressure on the back of the patient's feet.

It is thus desired to have a corrective foot orthosis device wherein a patient's feet are maintained at a desired angle while allowing the patient's legs to have independent mobility to potentially improve comfort and reduce undue pressure on the skin.

### SUMMARY

A further understanding of the functional and advantageous aspects of the disclosure can be realized by reference to the following detailed description and drawings.

A dynamic foot abduction bar for use with a pair of boots having a crossbar and a pair of generally L-shaped components. The generally L-shaped components are slidingly attached at either end of the crossbar, thereby allowing axial translation. The pair of boots are releasably attachable to the generally L-shaped components.

A dynamic foot abduction bar for use with a pair of boots having a crossbar and a pair of generally L-shaped components. The pair of generally L-shaped components are operably attached at either end of the crossbar configured for upward coronal rotation, outward axial rotation and sagittal rotation. The pair of generally L-shaped components are operably attached at either end of the crossbar configured for axial translation. The pair of boots are releasably attachable to the generally L-shaped components.

The dynamic foot abduction bar may include a modified ball joint attached between the generally L-shaped component and the crossbar.

The modified ball joint may be configured for upward coronal rotation, outward axial rotation and sagittal rotation.

A pivot attached to the crossbar may be configured for outward axial rotation.

The pivot attachment to the crossbar may be configured for sagittal rotation.

The pivot attachment to the crossbar may be configured for upward coronal rotation.

The dynamic foot abduction bar may include a modified ball joint pivotally attached between each generally L-shaped component and the crossbar wherein the modified ball joint is configured for upward coronal rotation, outward axial rotation and sagittal rotation.

Each generally L-shaped component may be sliding attached the crossbar using a roller assembly.

Each generally L-shaped component may have a generally vertical portion and a generally horizontal portion and wherein generally semicylindrical features are formed on either side of the generally vertical portion and the roller assembly rollingly attaches to the semicylindrical features.

The crossbar may be a telescoping crossbar.

The boots may be releasably attached to the generally L-shaped components with a quick release.

A modified ball joint includes a ball portion, a socket portion and an insert. The socket portion is configured to receive the ball portion and allow free rotation. The insert may be positioned in or adjacent to the socket portion and limits the range of motion of the ball portion.

The internal edge of the modified ball joint may be generally L-shaped.

### BRIEF DESCRIPTION OF THE DRAWINGS

Embodiments will now be described, by way of example only, with reference to the drawings, in which:

FIG. 1 is a perspective view of a dynamic foot abduction bar;

FIG. 2 is a generally front view of the dynamic foot abduction bar of FIG. 1;

FIG. 3 is a perspective view of a baby showing the planes and range of motion;

FIG. 4 is a top view of the dynamic foot abduction bar of FIG. 1 and FIG. 2;

FIG. 5 is a perspective view of the dynamic foot abduction bar of FIGS. 1, 2 and 4, but without the boots;

FIG. 6 is an enlarged perspective view of the generally L-shaped component and a portion of the roller assembly;

FIG. 7 is a top view of the dynamic foot abduction bar of FIGS. 1, 2, 4 to 6 showing the range of motion for axial rotation;

FIG. 8 is a front view the dynamic foot abduction bar of FIGS. 1, 2, 4 to 7 showing the range of motion for coronal rotation;

FIG. 9 is an enlarged perspective view of the ball joint and L-shaped foot bracket of the dynamic foot abduction bar of FIGS. 1, 2, 4 to 8;

FIG. 10 is an enlarged view of the ball joint shown in FIG. 9;

FIG. 11 is an enlarged blown apart view of the ball joint of FIG. 10;

FIG. 12 is a perspective view of an alternate embodiment of the dynamic foot abduction bar;

FIG. 13 is an enlarged perspective view of the generally L-shaped component and a portion of the roller assembly of the embodiment of FIG. 12;

FIG. 14 is an enlarged perspective view of the ball joint and L-shaped foot bracket of the dynamic foot abduction bar of FIGS. 12 and 13;

FIG. 15 is an enlarged view of the ball joint shown in FIG. 14; and

FIG. 16 is an enlarged blown apart view of the ball joint of FIG. 15.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein the term “operably connected” or “operably attached” means that the two elements are connected or attached either directly or indirectly. Accordingly, the items need not be directly connected or attached but may have other items connected or attached therebetween.

As used herein, the term “generally” is meant to cover variations that may exist in the upper and lower limits of the ranges of values, such as variations in properties, parameters, and dimensions. Unless otherwise specified, the terms “generally” means plus or minus 25 percent or less.

The following examples are presented to enable those skilled in the art to understand and to practice embodiments of the present disclosure. They should not be considered as a limitation on the scope of the disclosure, but merely as being illustrative and representative thereof.

The specific embodiments described herein have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

The device shown herein is a modified foot abduction bar to be used for bracing in clubfoot treatment. The bar seeks to reduce brace intolerance, which is a leading cause of relapse in clubfoot treatment.

Standard of care for treatment of clubfoot is the Ponseti method, which consists of serial casting and a percutaneous Achilles tenotomy to correct the foot shape. The correction is maintained using a foot abduction brace in early childhood. Typical wear schedule for the foot abduction brace is 23 hours per day for three months, then at night for four years. Ideal position for the clubfoot is 10 degrees of dorsiflexion, and 70 degrees of external rotation relative to the hip with the feet held shoulder-width apart. If only one foot is affected, the normal foot would be kept in 10 degrees of dorsiflexion, and 30 degrees of external rotation. The dynamic foot abduction bar shown herein allows the child to independently extend and flex their knees and hips while maintaining the corrected foot position with the aim of improving comfort while wearing the brace and therefore decreasing brace intolerance and relapse.

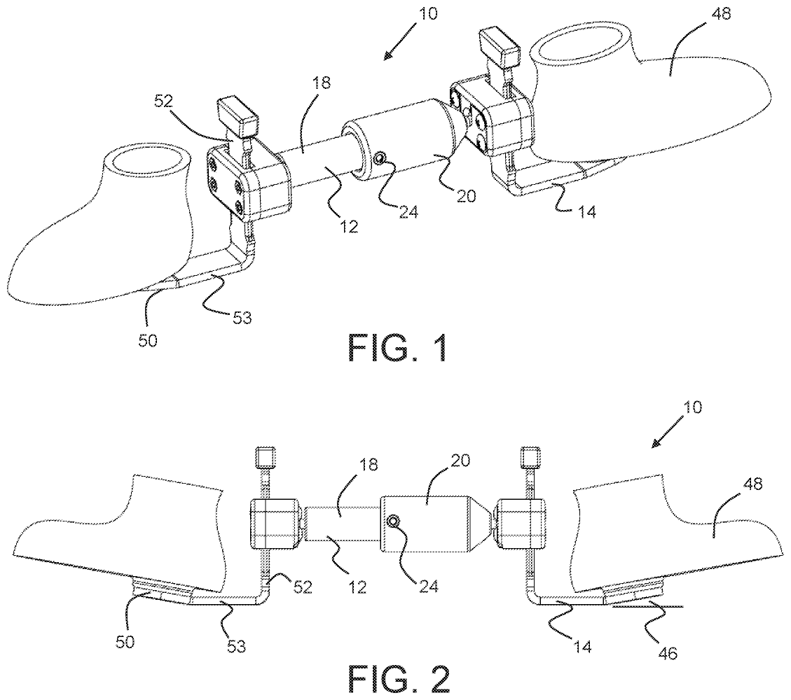

Referring to FIGS. 1 and 2, the dynamic foot abduction bar is shown generally at 10. The dynamic foot abduction bar 10 includes a crossbar 12 and a pair of generally L-shaped components 14 operably attached thereto. The crossbar 12 is adjustable. The crossbar 12 includes first and second tubes 18, 20 that are telescopingly attached together and secured with a set screw 24. The pair of generally L-shaped components 14 are pivotally attached and slidingly attached to the crossbar 12.

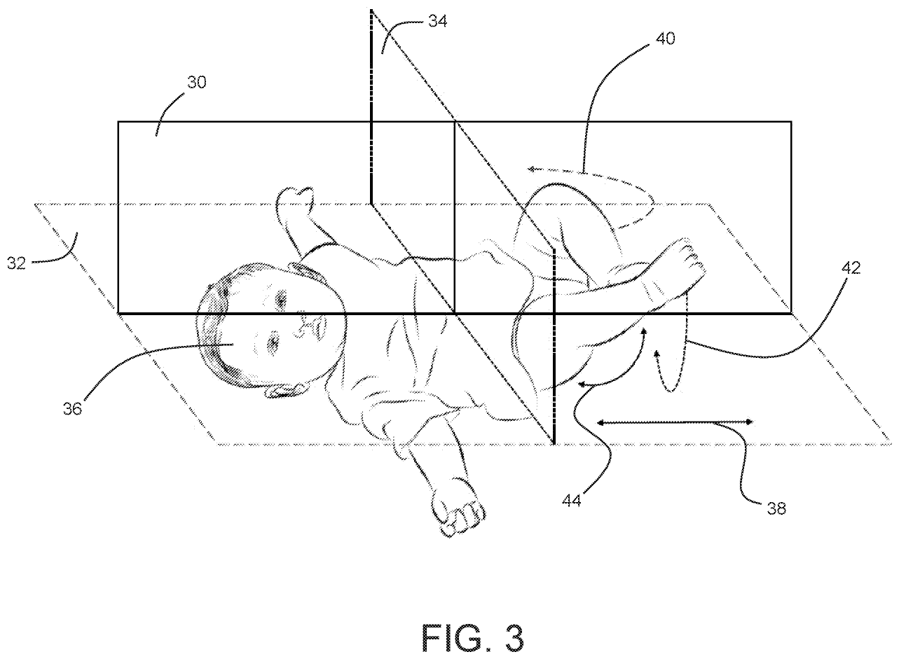

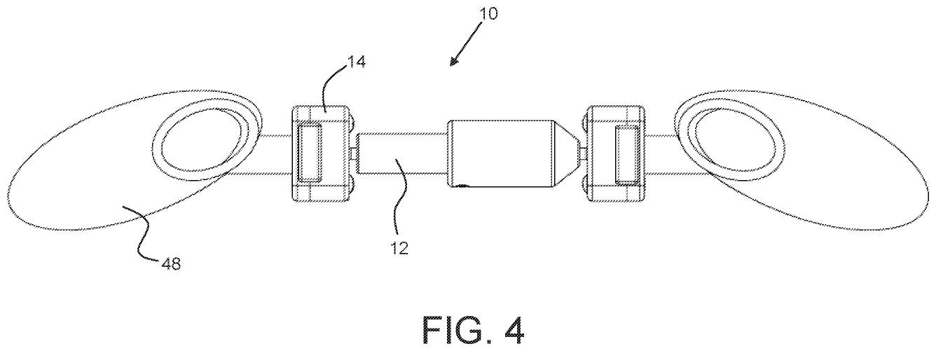

To provide context, FIG. 3 shows the different planes and rotational movement used to describe the range of motion. Note that the planes referred to herein are the axial, sagittal and coronal planes of the body. Specifically, the sagittal plane is shown at 30, the coronal plane at 32, and the axial plane at 34 for a child 36. The arrows show different rotational and translational movement. Axial translation is shown at 38. Coronal rotation is shown at 40, axial rotation is shown at 42, and sagittal rotation is shown at 44.

The generally L-shaped component 14 has a generally vertical portion 52 and a generally horizontal portion 53. The distal end of the generally horizontal portion is angled upwardly. Specifically, the foot position is maintained as each generally L-shaped component 14 has a fixed 10 degrees of ankle dorsiflexion as best seen in FIG. 2 at 46. A pair of boots 48 are releasably attachable to the generally horizontal portion 53 of the pair of L-shaped components 14. The minimum external rotation is variable and can be set by the user or medical practitioner.

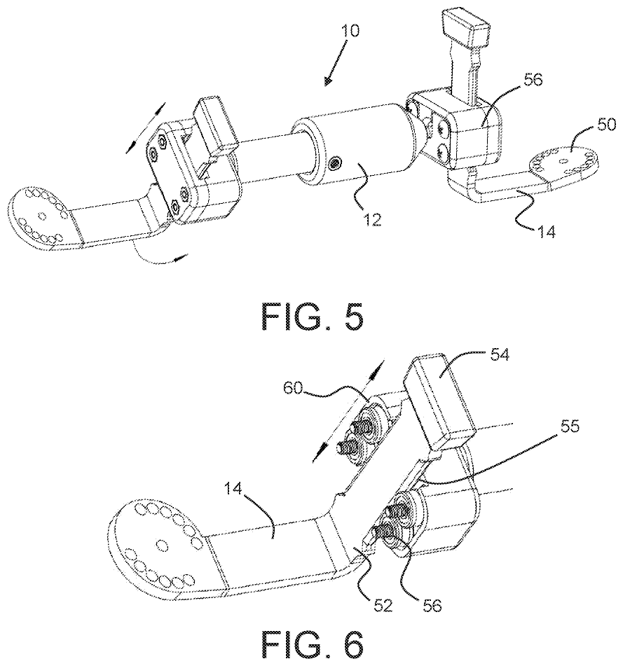

In the embodiment shown herein, the minimum external rotation is set using mating pin and hole attachment assembly 50 attached to the generally L-shaped component 14 and the boot 48. The hole portion of the mating pin and attachment assembly is best seen in FIG. 6. This position is set and not altered at any point during the range of motion afforded dynamic foot abduction bar 10. The mating pin and hole attachment assembly 50 may be part of the quick release clip shown in U.S. Pat. No. 10,675,170.

It will be appreciated by those skilled in the art that mating pin and hole attachment assembly 50 is by way of example only. Other arrangements for releasably attaching the pair of boots 48 to the pair of generally L-shaped components 14 respectively could also be used.

The generally L-shaped component 14 is operably connected to the crossbar 12 with a roller assembly. As best seen in FIGS. 5 and 6, the vertical portion 52 of the generally L-shaped component 14 has a pair of elongated semicylindrical features 55 on either side thereof. A roller assembly 56 is rollingly attached to the semicylindrical features 55 through a plurality of side rollers 60. The vertical portion is provided with an end cap 54 which may function as a stopper for the roller assembly 56.

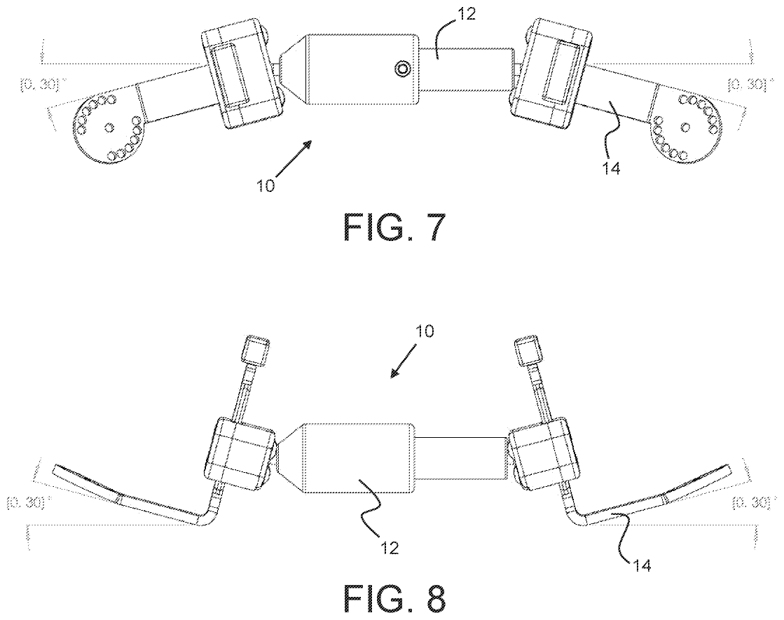

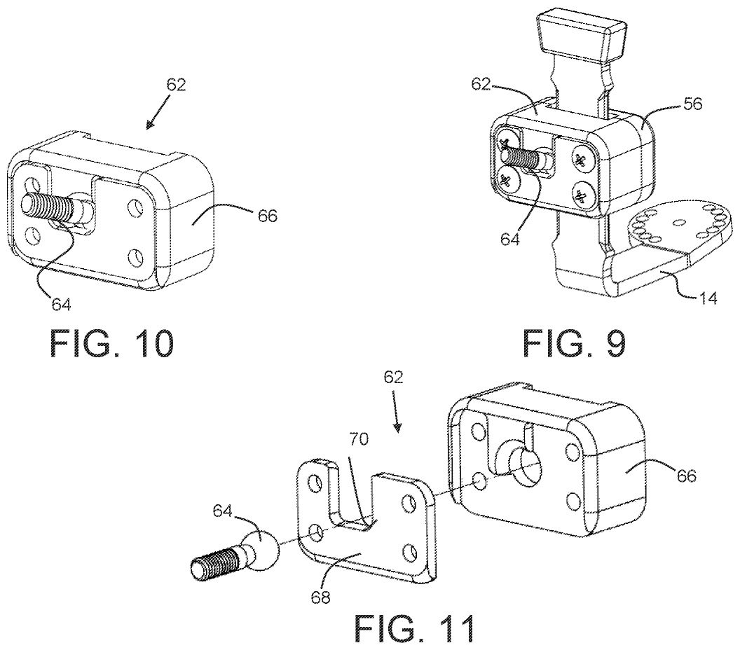

A pair of modified ball joint linkages 62 are attached to either ends of the crossbar 12. Referring to FIGS. 10 and 11, each modified ball joint linkage 62 includes a ball portion 64, a socket portion 66 and an insert 68 operably attached to the socket portion 66. The socket portion 66 is integrated into the roller assembly 56 and configured to receive the ball portion 64 and allow for free rotation within the socket. Insert 68 is positioned adjacent to the socket portion 66 and limit the range of motion in predetermined directions. Insert 68 has an internal edge 70 that limits the range of motion of the ball portion 64. As shown in FIG. 8 the generally L-shaped component 14 is configured for upward coronal rotation and as shown in FIG. 7 the generally L-shaped component 14 is configured for outward axial rotation. The range of rotation is defined by the size of the ball joint. In the embodiment shown herein the range of rotation is 30 degrees. It will be appreciated by those skilled in the art that different sized ball joints could be similarly adapted.

The modified ball joint linkages 62 provide a small amount of independent axial and coronal rotation. This enables the child to rotate their hips inwards and outwards to a maximum of predetermined amount in each direction. In the embodiment shown herein the predetermined amount is 30 degrees. This aims to enable more physiologic movement through the knee and hips, reduces strain at the child's joints, and reduces stress concentration at the hinges of the bar. The modified ball joint linkages 62 therefore reduce potential discomfort for the child and reduce the chance that the bar will break while in use. The crossbar 12 is adjustable in length as it consists of telescoping tubes 18, 20 that are set to the desired length using a set screw 24.

The extra mobility is achieved through a 3-part bar: a crossbar 12 with two generally L-shaped components 14 on each end that connect to the boots 48. The rollers 60 on the generally L-shaped components 14 allow independent axial translation perpendicular to the crossbar 12 (enabling hip flexion and extension). The modified ball joint linkages 62 connecting the generally L-shaped components 14 to the crossbar allow independent sagittal rotation (enabling knee flexion and extension). The dynamic foot abduction bar 10 allows the child to achieve almost full normal knee and hip motion. The dynamic foot abduction bar 10 can be scaled up as the child grows.

By providing children with clubfeet freedom to independently move their legs while maintaining the desired corrected foot position, this new dynamic foot abduction bar aims to improve patient comfort and reduce brace intolerance and clubfoot relapse. This new dynamic foot abduction bar design should lead to improved clinical outcomes of clubfoot, as well as patient and parent experience and satisfaction.

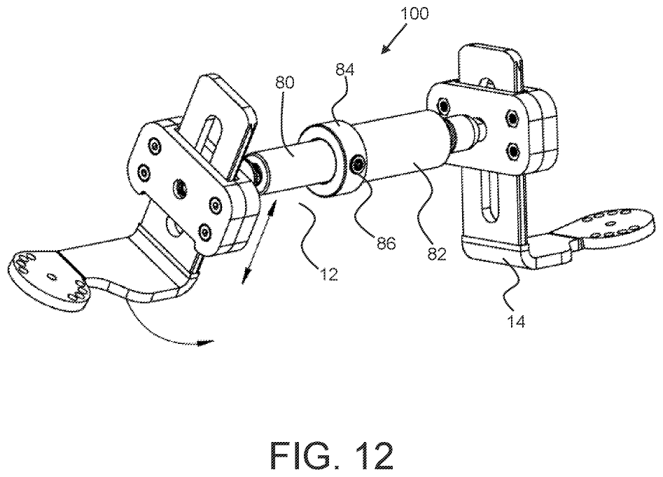

It will be appreciated by those skilled in the art that the dynamic foot abduction bar 10 described above could be modified while providing the same functionality. An alternate embodiment is shown in FIGS. 12 to 16. The alternate embodiment is shown generally at 100.

Referring to FIG. 12, as described above the dynamic foot abduction bar 100 includes a crossbar 12 and a pair of generally L-shaped components 14. The crossbar 12 is adjustable. The crossbar 12 includes first and second tubes 80, 82 that are telescopingly attached together and secured with a collar 84 and a set screw 86. In the embodiment shown herein collar 84 is integrally attached to tube 82. The pair of generally L-shaped components 14 are pivotally attached and slidingly attached to the crossbar 12.

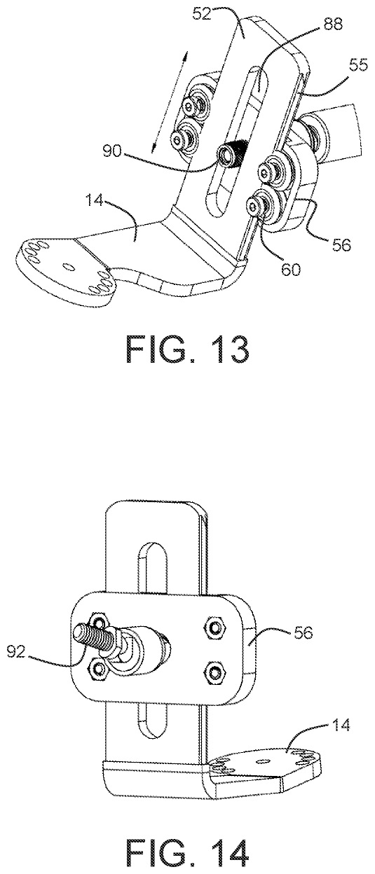

The generally L-shaped component 14 is connected to the crossbar 12 with a roller assembly. As best seen in FIG. 13 the vertical portion 52 of the generally L-shaped component 14 has a pair of elongated semicylindrical features 55 on either side thereof and a slot 88 down the middle. A roller assembly 56 is rollingly attached to the semicylindrical features 55 through a plurality of side rollers 60. The roller assembly 56 also includes a central pin 90 that slides upwardly and downwardly in the slot 88 relative to the generally L-shaped component 14.

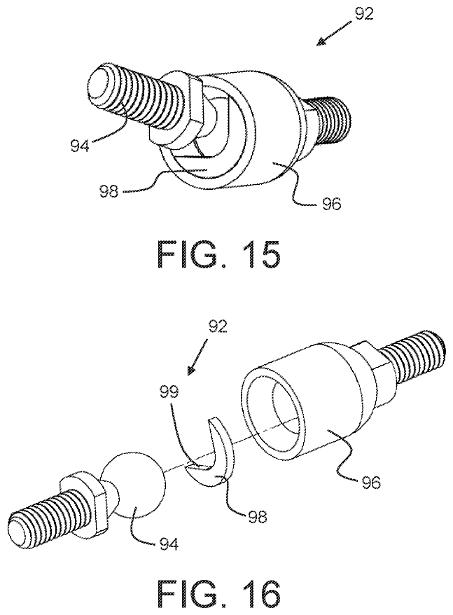

A pair of modified ball joint linkages 92 are attached to either ends of the crossbar 12. The modified ball joint linkage 92 is attached to central pin 90. Referring to FIGS. 15 and 16, each modified ball joint linkage 92 includes a ball portion 94, a socket portion 96 and an insert 98 operably attached to the socket portion 96. The socket portion 96 is configured to receive the ball portion 94 and allow for free rotation within the socket. The insert 98 is positioned in the socket portion 96 and limits the range of motion in predetermined directions. The insert 98 has an internal edge 99 that is generally L-shaped and the internal edge 99 limits the range of motion of the ball portion 94. The range of rotation is defined by the size of the ball joint. In the embodiment shown herein the range of rotation is 30 degrees. It will be appreciated by those skilled in the art that different sized ball joints could be similarly adapted.

1. A dynamic foot abduction bar for use with a pair of boots comprising:
a crossbar;
a pair of generally L-shaped components slidingly attached at either end of the crossbar, thereby allowing axial translation; and
the pair of boots being releasably attachable to the pair of generally L-shaped components respectively.

2. The dynamic foot abduction bar as claimed in claim 1 wherein each generally L-shaped component is operably attached to the crossbar configured for upward coronal rotation.

3. The dynamic foot abduction bar as claimed in claim 2 wherein each generally L-shaped component is pivotally attached to the crossbar.

4. The dynamic foot abduction bar as claimed in claim 1 wherein each generally L-shaped component is operably attached to the crossbar configured for outward axial rotation.

5. The dynamic foot abduction bar as claimed in claim 4 wherein each generally L-shaped component is pivotally attached to the crossbar.

6. The dynamic foot abduction bar as claimed in claim 1 wherein each generally L-shaped component is operably attached to the crossbar configured for sagittal rotation.

7. The dynamic foot abduction bar as claimed in claim 6 wherein each generally L-shaped component is pivotally attached to the crossbar.

8. The dynamic foot abduction bar as claimed in claim 1 further including a modified ball joint pivotally attached between each generally L-shaped component and the crossbar wherein the modified ball joint is configured for upward coronal rotation, outward axial rotation and sagittal rotation.

9. The dynamic foot abduction bar as claimed in claim 8 wherein the modified ball joint includes a ball portion and a socket portion and the socket portion includes an insert operably attached to the socket portion to limit a range of motion of the ball portion.

10. The dynamic foot abduction bar as claimed in claim 1 wherein each generally L-shaped component is slidingly attached the crossbar using a roller assembly.

11. The dynamic foot abduction bar as claimed in claim 10 wherein each generally L-shaped component has a generally vertical portion and a generally horizontal portion and wherein generally semicylindrical features are formed on either side of the generally vertical portion and the roller assembly rollingly attaches to the semicylindrical features.

12. The dynamic foot abduction bar as claimed in claim 1 wherein the crossbar is a telescoping crossbar.

13. The dynamic foot abduction bar as claimed in claim 1 wherein the boots are releasably attached to the pair of generally L-shaped component with a quick release.

14. A dynamic foot abduction bar for use with a pair of boots comprising:
a crossbar;
a pair of generally L-shaped components operably attached at either end of the crossbar configured for upward coronal rotation, outward axial rotation, and sagittal rotation; and
the pair of boots being releasably attachable to the pair of generally L-shaped components respectively.

15. The dynamic foot abduction bar as claimed in claim 14 wherein each generally L-shaped component is attached to the crossbar with a modified ball joint.

16. The dynamic foot abduction bar as claimed in claim 14 wherein the pair of generally L-shaped components operably attached at either end of the crossbar are further configured for axial translation.

17. A modified ball joint comprising:
a ball portion;
a socket portion configured to receive the ball portion and allow free rotation; and
an insert operably attached to the socket portion to limit a range of motion of the ball portion.

18. The modified ball joint as claimed in claim 17 wherein the insert has an internal edge that is generally L-shaped.
